(19) United States
(12) Patent Application Publication
(10) Pub. No.: US 2026/0340837 A1
(43) Pub. Date: Oct. 29, 2026

(54) AUTO CONTOURING USING ULTRASOUND DATA AND MODELING USING ULTRASOUND CONTOURS

(71) Applicant: Biosense Webster (Israel) Ltd., Yokneam (IL)

(72) Inventors: Roy Urman, Haifa (IL); Stanislav Goldberg, Haifa (IL); Richard Matthew, Haifa (IL); Yariv Amos, Tzorit (IL); Lior Weizman, Tel Aviv (IL); Liron Mizrahi, Kiryat Bialik (IL)

(21) Appl. No.: 19/193,776

(22) Filed: Apr. 29, 2025

(51) Int. Cl. G06T 7/13; A61B 6/00; A61B 8/00; A61B 8/08; A61B 8/12; G06T 7/136

(52) U.S. Cl. CPC G06T 7/13; A61B 6/5247; A61B 8/0883; A61B 8/466; A61B 8/5261; G06T 7/136; A61B 8/12; G06T 2207/10024; G06T 2207/10081; G06T 2207/10132; G06T 2207/20044; G06T 2207/20076; G06T 2207/20081; G06T 2207/20084; G06T 2207/30048

(57) ABSTRACT

A method for auto-contouring comprises receiving first data indicative of a characteristic of a first anatomical structure; training, based on at least the first data, a first learning model; receiving second data indicative of a characteristic of a second anatomical structure; and determining, based on the second data and the using the first learning model, a contour of the second anatomical structure.

### BACKGROUND

Conventional contouring of structures based on ultrasound data requires large amounts of ultrasound data, often with annotations marking specific areas of interest. Annotating may require the attention of a physician or multiple physicians. Gathering large amounts of ultrasound data may require an instrument to be inserted into a patient for a long period of time. It may also require skillful maneuvering by a physician. Improvements are needed.

### SUMMARY

It is to be understood that both the following general description and the following detailed description are exemplary and explanatory only and are not restrictive. Methods and systems for navigating an imaging device to capture images of an area of interest (e.g., region of an anatomical structure) are described.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, systems and methods are configured automatically detect contours of cardiac structures—LA (Left Atrium) body, LAA (Left Atrial Appendage), LIPV (Left Inferior Pulmonary Vein), LSPV (Left Superior Pulmonary Vein), RIPV (Right Inferior Pulmonary Vein), RSPV (Right Superior Pulmonary Vein)—using ultrasound data or images.

In another aspect, systems and methods automatically generate an anatomical model of anatomical area of interest (e.g., heart, a chamber or portion of the heart) based on ultrasound contours.

These and other features and advantages are described in greater detail below.

### BRIEF DESCRIPTION OF THE DRAWINGS

Some features are shown by way of example, and not by limitation, in the accompanying drawings. In the drawings, like numerals reference similar elements.

FIG. 1 shows an example user interface described herein.

FIG. 2 shows an example user interface described herein.

FIG. 3 shows an example user interface described herein.

FIG. 4 shows a flowchart of an example method disclosed herein.

FIG. 5 shows a flowchart of an example method disclosed herein.

FIG. 6 shows a flowchart of an example method disclosed herein.

FIGS. 7A-7D show stages of creating simulated ultrasound contours from an example 3D model.

FIGS. 8A-8D show voxelization and sampling of an example 3D model.

FIGS. 9A-9E show detail obtained from various sampling ratios of an example 3D model.

FIG. 10 shows a layout for an encoder and/or decoder disclosed herein.

FIG. 11 shows an example network architecture for a convolutional neural network (CNN) disclosed herein.

FIG. 12 shows a layout for an encoder and/or decoder disclosed herein.

FIG. 13 shows an example network architecture for a convolutional neural network (CNN) disclosed herein.

FIG. 14 shows an example training method disclosed herein.

FIG. 15 shows an example testing method disclosed herein.

FIG. 16 shows example ultrasound images.

FIG. 17 shows a flowchart of an example method disclosed herein.

FIG. 18 shows a flowchart of an example method disclosed herein.

FIG. 19 shows a flowchart of an example method disclosed herein.

The accompanying drawings show examples of the disclosure. It is to be understood that the examples shown in the drawings and/or discussed herein are non-exclusive and that there are other examples of how the disclosure may be practiced.

### DETAILED DESCRIPTION

The accompanying drawings, which form a part hereof, show examples of the disclosure. It is to be understood that the examples shown in the drawings and/or discussed herein are non-exclusive and that there are other examples of how the disclosure may be practiced.

The present disclosure relates to navigating an imaging device to capture images of an area of interest of an anatomical structure such as a left atrium of a heart, for example. Other areas or structures may be used. Reference is made throughout this disclosure to the left atrium of the heart as a non-limiting, illustrative example. Although conventional problems with imaging such structures as the left atrium of the heart may be addressed by the present disclosure, such references are not intended to limit the applicability of the systems and methods of the present disclosure to other structure or areas of anatomical structures. The description, figures, and functionality described herein, as applied to areas of the heart, may be applied to other areas and structures without departing from the spirit of the invention.

In certain cardiac imaging and reconstruction a 3-dimensional cardiac reconstruction is carried out by catheterizing a heart using a probe with a mapping electrode, and acquiring electrical data from respective locations in areas of interest in the heart, representing the locations of the electrical data as a point cloud, reconstructing a model of the heart from the point cloud, applying a set of filters to the model to produce a filtered volume, segmenting the filtered volume to define components of the heart, and reporting the segmented filtered volume, such as described in U.S. Pat. No. 9,265,434B2, which is hereby incorporated herein by reference in its entirety for any and all purposes. As a non-limiting example, such electroanatomical mapping (e.g., Fast Anatomical Mapping (FAM)) may be accomplished by the CARTO® 3 System cooperatively with a mapping catheter such as the Navistar® Thermocool® catheter, both available from Biosense Webster, Inc., 3333 Diamond Canyon Road, Diamond Bar, Calif. 91765. Other systems and catheters (e.g., mapping catheters) may be used in this manner.

As noted, segmentation may be required to separate a volume into the structure(s) of interest (SOI). In certain cases, the SOI is the area of most interest for real-time mapping, such as the left atrium or left ventricle (or other structures). In the context of cardiac imaging, segmentation of the left atrium may be necessary for various purposes, including volumetric analysis, functional assessment, and treatment planning, for example.

However, in certain applications imaging tools (e.g., ultrasound (ULS) catheters or systems) may be used where no or limited electroanatomical mapping is available. The present disclosure relates to systems and methods to generate an anatomical model (e.g., shell, 3D model) without the need of a diagnostic/mapping catheter, but instead using only the ultrasound data from an ultrasound catheter.

As a non-limiting example, one or more 3D models may be identified to include the entire left atrium (or at least a threshold amount of an area or structure of interest). From the one or more 3D models, one or more multi-plane images may be generated to visualize the left atrium from different angles. A model, such as a machine learning or probabilistic model may be developed (e.g., trained, tested, tuned) using the one or more multi-plane images as a representation of what target multi-plane images should look like when the entire area or structure of interest (e.g., left atrium) is successfully captured. As such, the model may then be implemented to provide feedback to users of whether inputted multi-plane images are satisfactory or not to capture a threshold portion of the left atrium.

As an example, in a real-time procedure, a user may capture one or more multi-plane images of the left atrium using a catheter such as a ULS catheter. Using the subject model, one may predict if the entire left atrium would be represented in the imaging field. If yes, then a 3D model may be generated including a threshold amount of the left atrium. If no, then instruction may be provided to instruct the user how to adjust the catheter in order to be in better position for viewing the threshold amount of the left atrium. Other structures of interest may be used.

As a further illustrative example, a plurality of three dimensional (3D) models may be received. The plurality of 3D models may each comprise a 3D model of a threshold amount of the area of interest of an anatomical structure such as the left atrium of a heart. The threshold amount may be 100%. Planar images may be generated from the plurality of 3D models. Multi-plane images may be generated from the plurality of 3D models. A multi-plane image may comprise two orthogonal planes. A generated multi-plane image may represent a multi-plane image an imaging instrument may have taken of an associated area of interest (e.g., left atrium) of an associated heart had the imaging instrument been used with an associated instrument position and an associated instrument orientation. The generated multi-plane images, associated instrument positions, and associated instrument orientations may be used to train an imaging model. The imaging model may take in a multi-plane image as input. The imaging model may use statistical probability to infer an instrument position and/or instrument orientation based on the input. The imaging model may use statistical probability infer if the imaging instrument comprises an instrument position and/or instrument orientation to capture sufficient imaging information to create a 3D model of an area of interest such as an associated left atrium of an associated heart.

The systems and/or methods disclosed herein may use feature extraction for model accuracy. Feature extraction may comprise using planar cross-sections, surface texture analysis, edge detection, etc. The systems and/or methods disclosed herein may use corrective feedback mechanisms for model completion. Corrective feedback mechanisms may comprise positional adjustments, orientation guidance, real-time alerts, etc. Using feature extraction and/or corrective feedback may improve an accuracy of left atrium imaging, reduce left atrium imaging procedure time, enhance overall clinical outcomes, etc. Other structures and areas of interest of the body may have associated features for extractions.

The systems and/or methods disclosed herein may use planar cross-sections for model accuracy. For example, the systems and/or methods disclosed herein may extract orthogonal planar cross-sections of the structure or area of interest to analyze coverage and completeness of the structure or area of interest within an imaging field. The extracted orthogonal planar cross-sections of the structure or area of interest may be analyzed to identify anatomical landmarks. For example, when the structure or area of interest is the left atrium of a heart, the anatomical landmarks may comprise pulmonary veins, a left atrial appendage, etc. Other anatomical landmarks may be used for the left atrium or other structures or areas of interest. Identification of the anatomical landmarks may confirm that an entire structure or area of interest is within the imaging field.

The systems and/or methods disclosed herein may use surface texture analysis for model accuracy. For example, a model may be examined for texture and contour of a surface of a structure or area of interest, such as a left atrium surface. A comparison may be performed between the examined texture and contour of the surface of the structure or area of interest and known templates of complete and/or incomplete models of structures or areas of interest. Based on the comparison, the systems and/or methods described herein may identify missing sections of the structure or area of interest and/or sections of the structure or area of interest that were poorly captured by the model. The systems and/or methods disclosed herein may prompt corrective action based on the missing sections and/or sections that were poorly captured.

The systems and/or methods disclosed herein may use edge detection algorithms to highlight boundaries of a structure or area of interest, such as a left atrium, in a model. Highlighting the boundaries of the structure or area of interest in the model helps a user ensure that the structure or area of interest is sufficiently captured, which helps ensure that the model is complete. Missing and/or unclear edges in the model may trigger feedback for the user to adjust a position and/or orientation of a catheter.

The systems and/or methods disclosed herein may use positional adjustments for model completion. The systems and/or methods described herein may extract features from a model, as explained above. If the extracted features indicate an incomplete capture of a structure or area of interest, the systems and/or methods may provide specific feedback on how to reposition an imaging instrument. For example, the systems and/or methods disclosed herein may provide feedback indicating a particular rotation (e.g., clockwise, slightly clockwise, clockwise 15 degrees) or lateral movement (e.g., forward, slightly forward, forward 2 millimeters) to ensure imaging of all areas of interest of the structure or area, such as a left atrium.

The systems and/or methods disclosed herein may use orientation guidance for model completion. The systems and/or methods described herein may guide a user in adjusting a tilt or orientation of an imaging instrument to better align with a structure of a left atrium. Structures of the left atrium that may be of value include a mitral valve, pulmonary veins, etc. The systems and/or methods described herein may provide guidance (e.g., deflect to the left, deflect slightly to the left, deflect 5 degrees to the left) to obtain clear imaging of the structures of the left atrium. Other structures or areas of interest may be used.

The systems and/or methods disclosed herein may use real-time alerts for model completion. During an imaging process, the systems and/or methods described herein may detect whether a structure or area of interest, such as a left atrium, is captured sufficiently. If the systems and/or methods described herein detect that the structure or area of interest is captured insufficiently, then the systems and/or methods described herein may generate real-time alerts to prompt a user to make corrections. Using real-time alerts may eliminate a need for repeat scan and enhance an accuracy of a final 3D model.

The systems and/or methods described herein may determine that a left atrium appendage is not fully visible in a current multi-plane image. Other structures or areas of interest may be used. The systems and/or methods described herein may create feedback advising adjustment an imaging instrument by rotating the imaging instrument 20 degrees counter-clockwise.

The systems and/or methods described herein may determine that pulmonary veins are only partially captured. Other structures or areas of interest may be used. The systems and/or methods described herein may create feedback suggesting retraction of an imaging instrument slightly and increasing a tilt angle of the imaging instrument by 7 degrees to improve visibility.

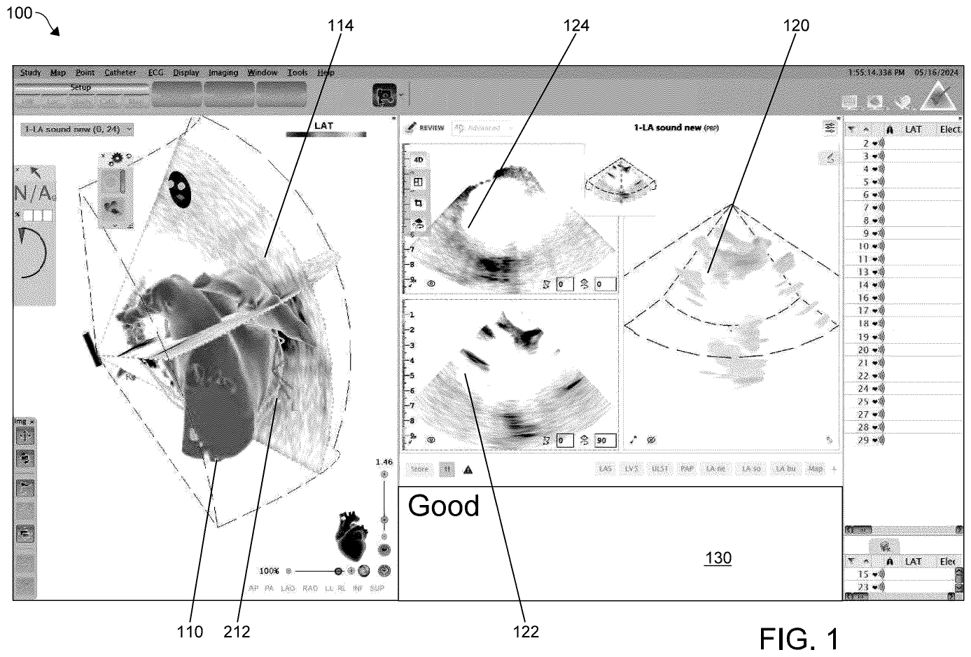

FIG. 1 shows an example user interface 100 described herein. The user interface may be associated with an imaging system such as the CARTO® 3 System by BIOSENSE WEBSTER, INC. Other systems and user interfaces may be used. One or more of a 3D model 110, a first planar image 112, a second planar image 114, a first image 122, a second image 124, or an imagining field 120 may be presented via the user interface 100. As an example, the user interface 100 may present one or more of the 3D model 110, the first planar image 112, the second planar image 114, the first image 122, the second image 124, or the imagining field 120 over time. The outputs of the user interface 100 may update in or near real-time.

The 3D model may be an anatomical model or shell that represents an imaged portion of an anatomical structure. As shown, the 3D model 110 comprises an overlayed first planar image 112 and second planar image 114. The first planar image 112 may be orthogonal to the second planar image 114. The first planar image 112 and the second planar image 114 may be created by an imaging instrument such as an intravascular ultrasound catheter. The imaging instrument may be configured to capture image data from inside a structure or area of interest, such as a heart, such as inside or adjacent a left atrium of the heart. The first planar image 112 may be based on a position and/or orientation of the imaging instrument. The second planar image 114 may be based on the position and/or orientation of the imaging instrument. The first planar image 112 and/or the second planar image 114 may be used to create the 3D model 110.

As described herein, the first planar image 112 and/or the second planar image 114 may be representative of multi-plane images that may be generated from the data of the 3D model 110 to visualize an area or structure of interest (e.g., the left atrium) from different angles. As further described herein, the 3D model 110 may represent one of a plurality of 3D models from which the one or more multi-plane images may be generated to visualize the structure or area of interest from different angles. A model, such as a machine learning or probabilistic model may be developed (e.g., trained, tested, tuned) using the one or more multi-plane images (e.g., the first planar image 112 and/or the second planar image 114) as a representation of what target multi-plane images should look like when the entire structure or area of interest is successfully captured. As an example, the model (e.g., neural network) may be configured to use feature extraction from multi-plane slices of the 3D models (e.g. Biplane or Biplane slices from 4D). As such, the model may then be implemented to provide feedback to users of whether inputted multi-plane images are satisfactory or not to capture a threshold portion of the structure or area of interest.

Various 3D models such as the example 3D model 110 may be stored. Information included in the 3D models or captured as reference data to the 3D models may be indicative of various multi-planar images taken at various positions and orientations. Various multi-planar images such as the first planar image 112 and/or the second planar image 114 may be stored. Information includes in the multiplanar images may include one or more of position or orientation. Such example 3D models and multi-planar images may be used as the knowledge base (e.g., training or testing set) to develop a model described herein. Other sources of imaging data for developing the model may be used.

While the first planar image 112 and/or the second planar image 114 may be represented overlaying the 3D model 110, information represented by the first planar image 112 may be displayed in first image 122 and information represented by the second planar image 114 may be displayed in second image 124.

Presented another way, a user may capture one or more multi-plane images of the structure or area of interest using a catheter such as a ULS catheter. As an example, a user interface such as the user interface 100 may be configured to output a representation of the imaging data captured by the catheter. As a further example, first image 122 and the second image 124 may represent ULS data captured by the catheter from various orientations or positions. The data captured to generate one or more of the first image 122 and the second image 124 may be used to generate an imaging field 120, which may be configured to present a 4D representation of the ULS data showing 3D imagery data over time. Other images may be used.

Using the subject model, one may predict if the threshold amount of the structure or area of interest would be represented in the imaging field 120 as the user captures the one or more multi-plane images. As an illustrative example, the imaging field 120 may represent a 3D volume of an area or structure over time (e.g., 4D volume) and the % coverage of the structure or area of interest may be determined from the volume represented in the imaging field 120. If the threshold amount of the structure or area of interest is represented in the 3D volume in the imaging field 120, then a 3D model may be generated including a threshold amount of the structure or area of interest. If the threshold amount of the structure or area of interest is not represented in the 3D volume in the imaging filed 120, then instruction may be provided to instruct the user how to adjust the catheter in order to be in better position for viewing the threshold amount of the structure or area of interest.

For example, the user interface 100 may comprise a feedback message 130. The feedback message 130 may comprise a message regarding corrective positioning and/or corrective orientation of the imaging device. For example, the feedback message 130 may indicate that the imaging device should be deflected away from a septum. The 3D model 110 may comprise at least a portion of the structure or area of interest. The feedback message 130 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

As shown, for example, in FIG. 1, the feedback message 130 may comprise a message confirming a satisfactory position and/or satisfactory orientation of the imaging device. For example, the feedback message 130 may indicate that the imaging device comprises a good position and/or good orientation. The 3D model 110 may comprise at least a portion of the structure or area of interest. The feedback message 130 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied.

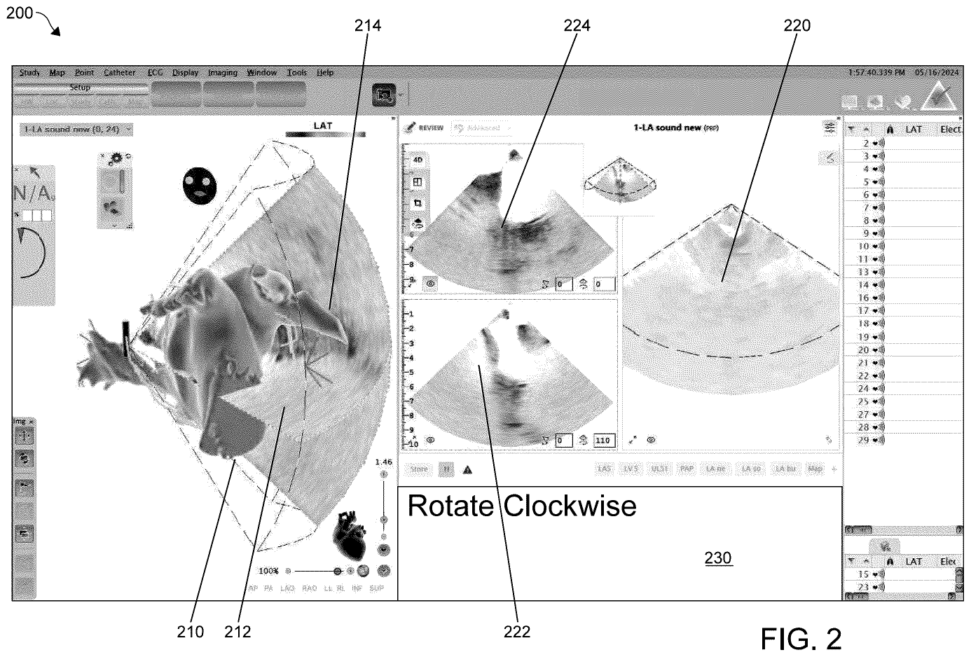

FIG. 2 shows an example user interface 200 described herein. The user interface 200 may similar elements to the user interface 100 in FIG. 1. The user interface 200 may comprise a 3D model 210 comprising a first planar image 212 and a second planar image 214, a first image 222 corresponding to the first planar image 212, a second image 224 corresponding to the second planar image 214, a visual display 220 of the 3D model 210 changing over time, and a feedback message 230. The feedback message 230 may a comprise message regarding corrective positioning and/or corrective orientation of the imaging device. For example, the feedback message 230 may indicate that the imaging device should be rotated clockwise. The 3D model 210 may comprise at least a portion of the structure or area of interest, such as the LA (Left Atrium) body, LAA (Left Atrial Appendage), LIPV (Left Inferior Pulmonary Vein), LSPV (Left Superior Pulmonary Vein), RIPV (Right Inferior Pulmonary Vein), RSPV (Right Superior Pulmonary Vein) of the heart. The feedback message 230 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

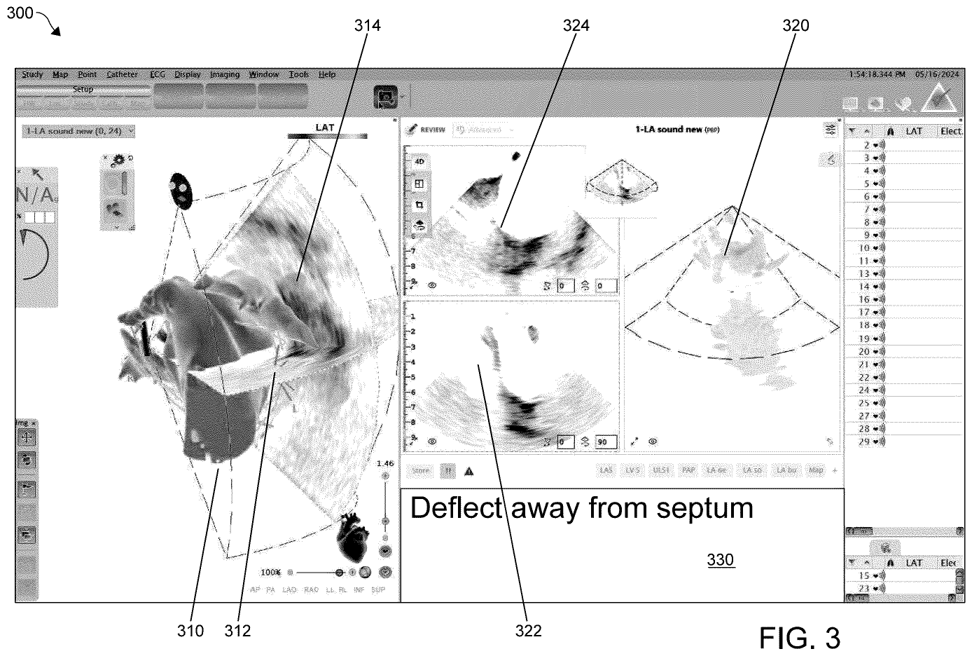

FIG. 3 shows an example user interface 300 described herein. The user interface 300 may similar elements to the user interface 100 in FIG. 1 and the user device 200 in FIG. 2. The user interface 300 may comprise a 3D model 310 comprising a first planar image 312 and a second planar image 314, a first image 322 corresponding to the first planar image 312, a second image 324 corresponding to the second planar image 314, a visual display 320 of the 3D model 310 changing over time, and a feedback message 330. The user interface 300 may show elements after instructions regarding the imagining instructions in the feedback message 230 in FIG. 2 are followed. The feedback message 330 may a comprise message regarding corrective positioning and/or corrective orientation of the imaging device. For example, the feedback message 330 may indicate that the imaging device should be deflected away from the septum. The 3D model 310 may comprise at least a portion of the structure or area of interest, such as the LA (Left Atrium) body, LAA (Left Atrial Appendage), LIPV (Left Inferior Pulmonary Vein), LSPV (Left Superior Pulmonary Vein), RIPV (Right Inferior Pulmonary Vein), RSPV (Right Superior Pulmonary Vein) of the heart. The feedback message 330 may comprise a color background indicative of if the at least portion of the structure or area of interest satisfies a threshold amount of the structure or area of interest. For example, a red or yellow colored background may indicate that the threshold amount is not satisfied. For example, a blue or green colored background may indicate that the threshold amount is satisfied. The message regarding corrective positioning and/or corrective orientation of the imaging device may comprise instructions intended to put the imaging instrument in a position to take a multi-planar image that may create a 3D model that satisfies the threshold amount.

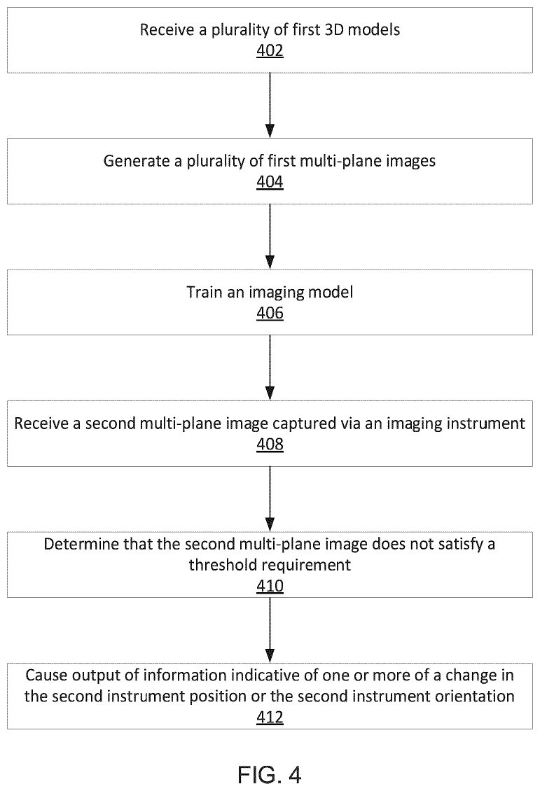

FIG. 4 is a flowchart of an example process 400. In some implementations, one or more process blocks of FIG. 4 may be performed by a computing device. The computing device may create the images shown in FIGS. 1-3. The computing device may be in communication with a second computing device, wherein the second computing device may create the images shown in FIGS. 1-3.

As shown in FIG. 4, process 400 may include receiving a plurality of first three dimensional (3D) models (block 402). For example, a computing device may receive a plurality of first 3D models. Each of the first 3D models may include a threshold amount of a structure or area of interest, such as the LA (Left Atrium) body, LAA (Left Atrial Appendage), LIPV (Left Inferior Pulmonary Vein), LSPV (Left Superior Pulmonary Vein), RIPV (Right Inferior Pulmonary Vein), RSPV (Right Superior Pulmonary Vein) of the heart. The threshold amount may comprise 100%. As a further example, the first 3D models may comprise a training set or testing set of data relating to the development of a probabilistic model or learning algorithm. The first 3D models may comprise ULS 3D data and/or anatomical mapping (e.g., FAM, CT, or the like).

As shown in FIG. 4, process 400 may include generating a plurality of first multi-plane images (block 404). For example, the computing device may generate a plurality of first multi-plane images. The plurality of first multi-plane images may be generated based at least on a portion of the plurality of first 3D models. Each of the first multi-plane images may include a first instrument position and a first instrument orientation.

As shown in FIG. 4, process 400 may include training an imaging model (block 406). For example, the computing device may train an imaging model. The imaging model may be trained based at least on the plurality of first multi-plane images.

As shown in FIG. 4, process 400 may include receiving a second multi-plane image captured via an imaging instrument (block 408). For example, the computing device may receive a second multi-plane image captured via an imaging instrument. The imaging instrument may have a second instrument position and a second instrument orientation. The receiving a second multi-plane image captured via an imaging instrument may comprise receiving the second multi-plane image in or near real-time as the second multi-plane image is captured via the imaging instrument. The imaging instrument may be configured to emit ultrasound waves.

As shown in FIG. 4, process 400 may include determining that the second multi-plane image does not satisfy a threshold requirement (block 410). For example, the computing device may determine that the second multi-plane image does not satisfy a threshold requirement. Determining that the second multi-plane image does not satisfy a threshold requirement may be based at least on the second multi-plane image and the imaging model. The threshold requirement may be and/or comprise a threshold requirement to generate a second 3D model having the threshold amount. The determining that the second multi-plane image does not satisfy a threshold requirement may comprise determining that the second multi-plane image does not have sufficient planar information for the imaging model to generate a full 3D model.

As shown in FIG. 4, process 400 may include causing output of information indicative of one or more of a change in the second instrument position or the second instrument orientation (block 412). For example, the computing device may cause output of information indicative of one or more of a change in the second instrument position or the second instrument orientation. The output may be caused based at least on the second multi-plane image and the imaging model. The information indicative of one or more of a change in the second instrument position or the second instrument orientation may be configured to result in a capture of a third multi-plane image having the threshold amount.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

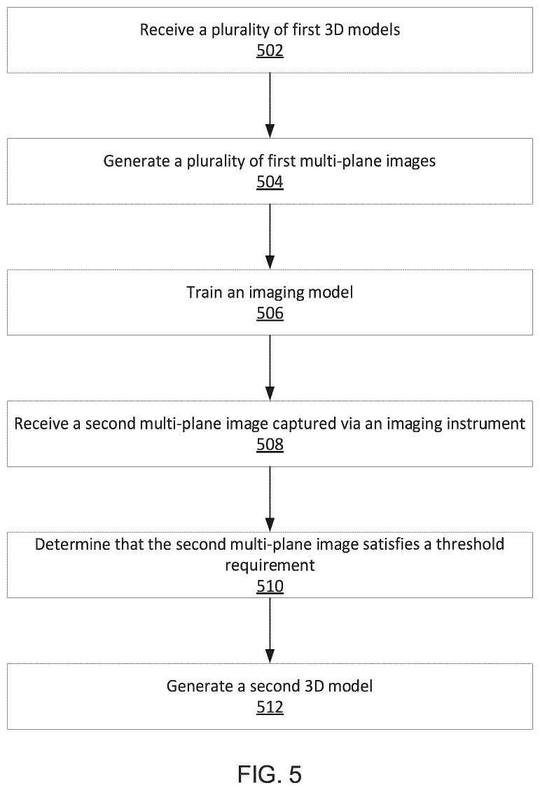

FIG. 5 is a flowchart of an example process 500. In some implementations, one or more process blocks of FIG. 5 may be performed by a computing device. The computing device may create the images shown in FIGS. 1-3. The computing device may be in communication with a second computing device, wherein the second computing device may create the images shown in FIGS. 1-3.

As shown in FIG. 5, process 500 may include receiving a plurality of first three dimensional (3D) models (block 502). For example, a computing device may receive a plurality of first 3D models. Each of the first 3D models may include a threshold amount of a structure or area of interest, such as the LA (Left Atrium) body, LAA (Left Atrial Appendage), LIPV (Left Inferior Pulmonary Vein), LSPV (Left Superior Pulmonary Vein), RIPV (Right Inferior Pulmonary Vein), RSPV (Right Superior Pulmonary Vein) of the heart. The threshold amount may comprise 100%.

As shown in FIG. 5, process 500 may include generating a plurality of first multi-plane images (block 504). For example, the computing device may generate a plurality of first multi-plane images. The plurality of first multi-plane images may be generated based at least on a portion of the plurality of first 3D models. Each of the first multi-plane images may include a first instrument position and a first instrument orientation.

As shown in FIG. 5, process 500 may include training an imaging model (block 506). For example, the computing device may train an imaging model. The imaging model may be trained based at least on the plurality of first multi-plane images.

As shown in FIG. 5, process 500 may include receiving a second multi-plane image captured via an imaging instrument (508). For example, the computing device may receive a second multi-plane image captured via an imaging instrument. The imaging instrument may have a second instrument position and a second instrument orientation. The receiving a second multi-plane image captured via an imaging instrument may comprise receiving the second multi-plane image in or near real-time as the second multi-plane image is captured via the imaging instrument. The imaging instrument may be configured to emit ultrasound waves.

As shown in FIG. 5, process 500 may include determining that the second multi-plane image satisfies a threshold requirement (block 510). The computing device may determine that the second multi-plane image satisfies a threshold requirement. Determining that the second multi-plane image satisfies a threshold requirement may be based at least on the second multi-plane image and the imaging model. The threshold requirement may be and/or comprise a threshold requirement to generate a second 3D model having the threshold amount. The determining that the second multi-plane image satisfies a threshold requirement may comprise determining that the second multi-plane image has sufficient planar information for the imaging model to generate a full 3D model.

As shown in FIG. 5, process 500 may include generating a second 3D model (block 512). For example, the computing device may generate a second 3D model. The 3D model may be generated based on the second multi-plane image.

Although FIG. 5 shows example blocks of process 500, in some implementations, process 500 may include additional blocks, fewer blocks, different blocks, or differently arranged blocks than those depicted in FIG. 5. Additionally, or alternatively, two or more of the blocks of process 500 may be performed in parallel.

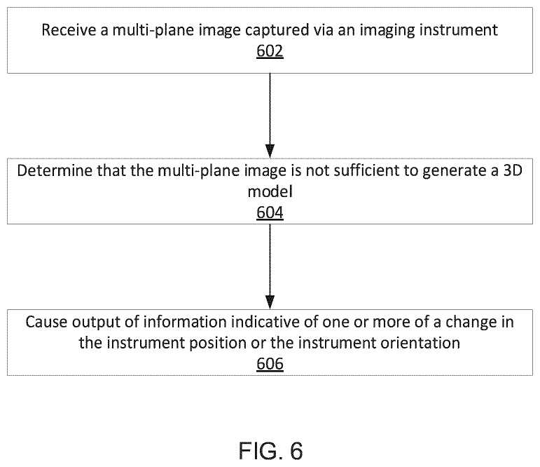

FIG. 6 is a flowchart of an example process 600. In some implementations, one or more process blocks of FIG. 6 may be performed by a computing device. The computing device may create the images shown in FIGS. 1-3. The computing device may be in communication with a second computing device, wherein the second computing device may create the images shown in FIGS. 1-3.

As shown in FIG. 6, process 600 may include receiving a multi-plane image captured via an imaging instrument (block 602). For example, a computing device may receive a multi-plane image captured via an imaging instrument. The imaging instrument may have an instrument position and an instrument orientation. The imaging instrument may be configured to emit ultrasound waves

As shown in FIG. 6, process 600 may include determining that the multi-plane image is not sufficient to generate a three dimensional (3D) model (block 604). For example, the computing device may determine that the multi-plane image is not sufficient to generate a 3D model. The determining that the multi-plane image is not sufficient to generate a 3D model may be based at least on the multi-plane image and an imaging model. The determining that the multi-plane image is not sufficient to generate a 3D model may comprise determining that the multi-plane image is not sufficient to generate a 3D model having a threshold amount of a structure or area of interest, such as a left atrium of a heart. The imaging model may be trained using a plurality of first multi-plane images. The plurality of first multi-plane images may comprise an associated instrument position and an associated instrument orientation. The plurality of first multi-plane images may have been generated based on a plurality of 3D models. The plurality of 3D models may comprise an associated threshold amount of an associated structure or area of interest. The threshold amount may be 100%.

As shown in FIG. 6, process 600 may include causing output of information indicative of one or more of a change in the instrument position or the instrument orientation (block 606). For example, the computing device may cause output of information indicative of one or more of a change in the instrument position or the instrument orientation. The output may be caused based at least on the multi-plane image and the imaging model. The information indicative of one or more of a change in the instrument position or the instrument orientation may be configured to result in a capture of another multi-plane image sufficient to generate a 3D model having the threshold amount.

Process 600 may comprise generating a new 3D model comprising the threshold amount of the structure or area of interest. For example, the computing device may generate a new 3D model comprising the threshold amount of the structure or area of interest. Process 600 may comprise producing at least one new multi-plane image based on the new 3D model. For example, the computing device may produce at least one new multi-plane image based on the new 3D model. Process 600 may comprise determining, based on the at least one new multi-plane image, if the determining that the multi-plane image is not sufficient to generate a 3D model comprising a threshold amount of the structure or area of interest was correct. For example, the computing device may determine, based on the at least one new multi-plane image, if the determining that the multi-plane image is not sufficient to generate a 3D model comprising a threshold amount of the structure or area of interest was correct.

Process 600 may include determining, based on the at least one new multi-plane image, that the determining that the multi-plane image is not sufficient to generate a 3D model comprising a threshold amount of the structure or area of interest was incorrect. For example, the computing device may determine, based on the at least one new multi-plane image, that the determining that the multi-plane image is not sufficient to generate a 3D model comprising a threshold amount of the structure or area of interest was incorrect. Process 600 may include updating the imaging model based on the new 3D model and the at least one new multi-plane image. For example, the computing device may update the imaging model based on the new 3D model and the at least one new multi-plane image.

Although FIG. 6 shows example blocks of process 600, in some implementations, process 600 may include additional blocks, fewer blocks, different blocks, or differently arranged blocks than those depicted in FIG. 6. Additionally, or alternatively, two or more of the blocks of process 600 may be performed in parallel.

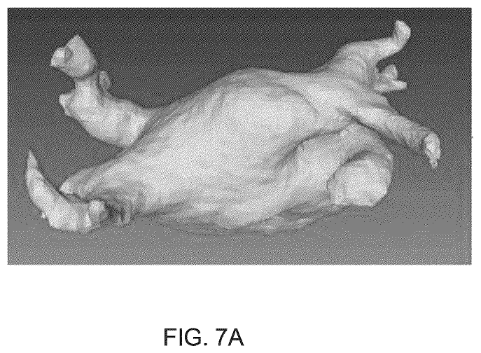

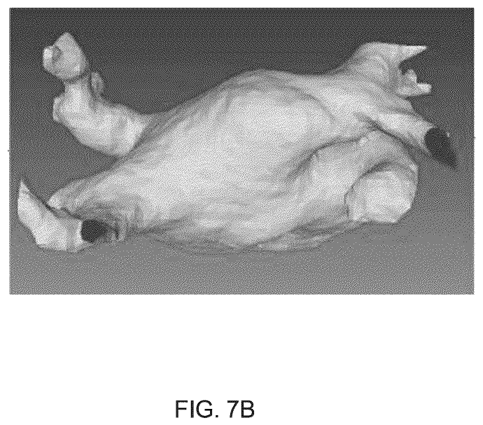

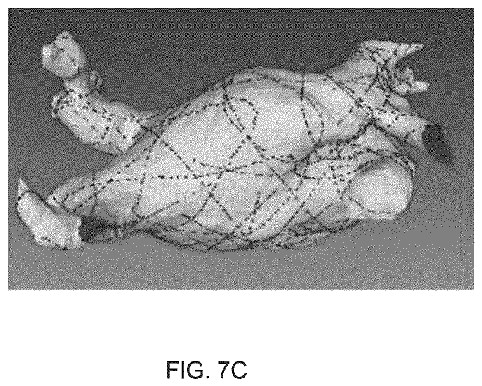

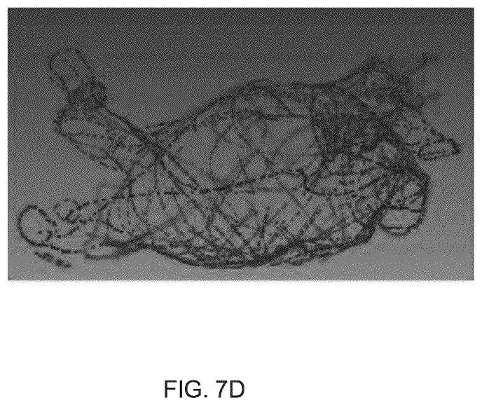

The systems and/or methods described herein may automatically generate an anatomical model of a structure or area of interest, such as a heart, or a portion of a heart, such as a chamber, such as a left atrium of a heart, based on ultrasound contours. A model creation algorithm may be created by using deep-learning training to associate simulated ultrasound contours with three-dimensional (3D) models. 3D models may comprise 3D images from computer topography (CT) scans or from ultrasound data. The stages of creating simulated ultrasound contours may be illustrated in FIGS. 7A-7D. FIG. 7A shows an existing 3D model of a heart, which may have been acquired via known methods (e.g., CT scans). FIG. 7B shows the model of FIG. 7A after the model has been trimmed. The model may be trimmed by a predetermined amount in each axis. The predetermined amount may be 5%. Trimming the model may remove a pulmonary veins region not sampled in an ultrasound contour.

FIG. 7C shows a number of two-dimensional (2D) slice (contour, plane, etc.) through the 3D model. The 2D slice may comprise a contour of the 3D model, such as a contour portion of the CT image. The 2D planes may be generated at random angles. The 2D slice may simulate contours acquired by an ultrasound instrument. The number of 2D slices shown in FIG. 7C is 35, although other numbers of 2D slices may be used, as explained below. FIG. 7D shows a final sampling. The final sampling may be a result of sampling a percentage of a surface of the 3D model. The percentage may be 80% or other percentage. The final sampling of each slice may simulate a field of view for an ultrasound instrument. FIG. 7D may simulate 35 ultrasound contours taken at random angles, such as those that may be expected in real-time use. FIG. 7D may be given as example input for the deep-learning training, and FIG. 7A and/or FIG. 7B may be given as example associated expected output for the example input.

The systems and/or methods described herein may perform voxelization of the contours to generate a 3D binary matrix. The voxelization may be performed because deep-learning may perform better with images and matrices as opposed to 3D meshes. A voxelization resolution may be set. For example, the voxelization resolution may be set to 0.5 millimeters. The voxelization resolution may be set based on computational resource considerations, such as graphics processing unit (GPU) memory limitations.

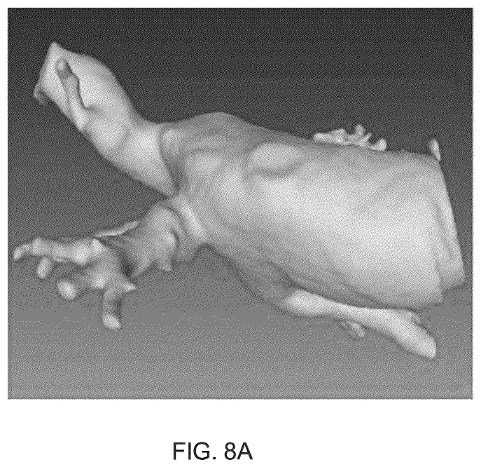

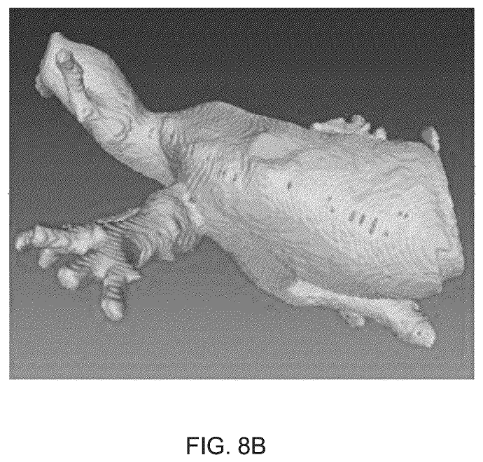

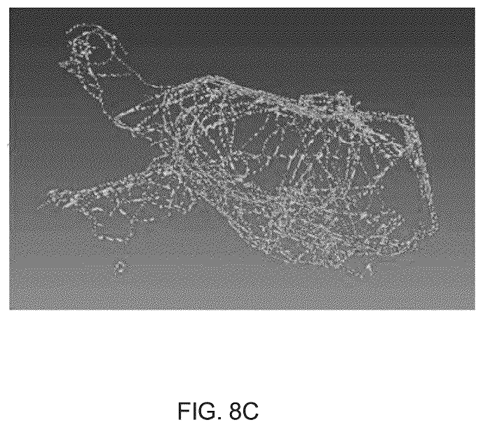

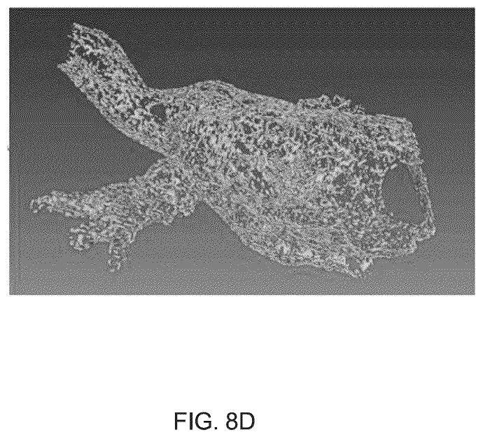

FIG. 8A shows an example 3D model from a CT scan. FIG. 8B shows an example voxelized model based on the 3D model in FIG. 8A. FIG. 8C shows a result of an example sampling of the voxelized model in FIG. 8B. The example sampling of FIG. 8C may represent 10% sampling ratio. The sampling ratio may be computed as a number of voxels used to represent the samplings in FIG. 8C divided by a number of voxels used to represent the voxelized model in FIG. 8B. FIG. 8D shows a result of an example sampling of the voxelized model in FIG. 8B. The example sampling of FIG. 8D may represent 30% sampling ratio. The sampling ratio may be computed as a number of voxels used to represent the samplings in FIG. 8D divided by a number of voxels used to represent the voxelized model in FIG. 8B.

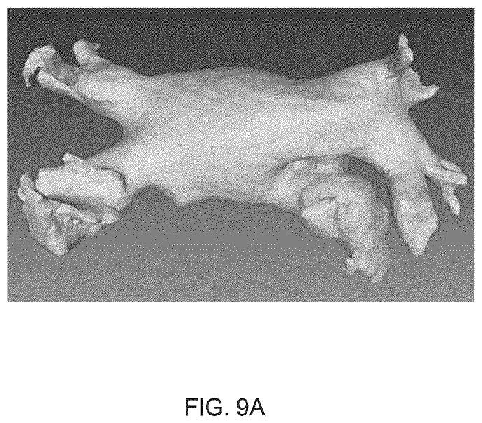

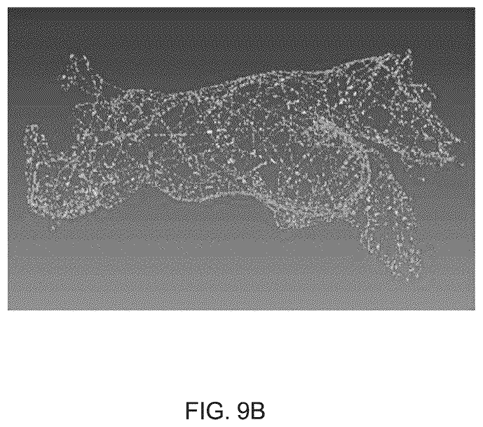

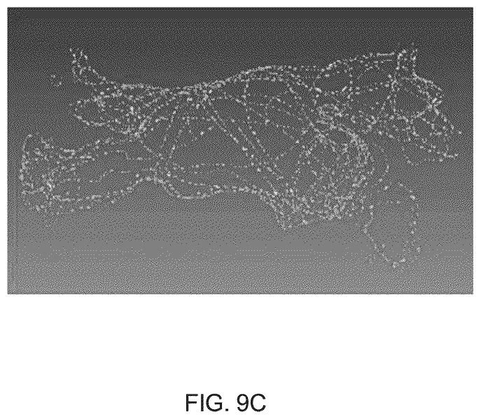

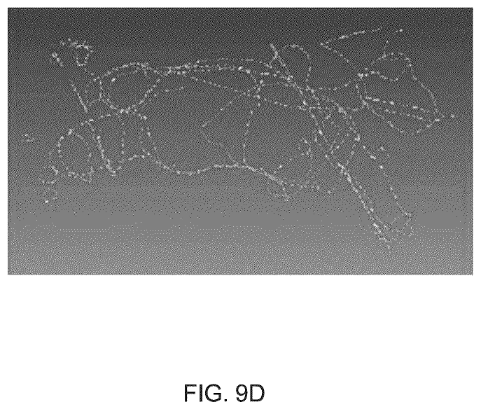

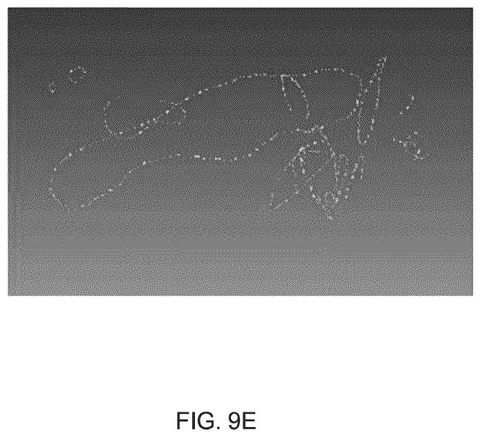

FIG. 9A shows an example 3D model from a CT scan. FIGS. 9B-9E demonstrate detail obtained from various sampling ratios of the 3D model in FIG. 9A. FIG. 9B shows 10% sampling, or about 50 planes (slices, etc.). FIG. 9C shows 5% sampling, or about 25 planes. FIG. 9D shows 3% sampling, or about 15 planes. FIG. 9E shows 1% sampling, or about 5 planes. Sampling ratios that replicate actual ultrasound instrument contours obtained in practice are about 3% to 5%.

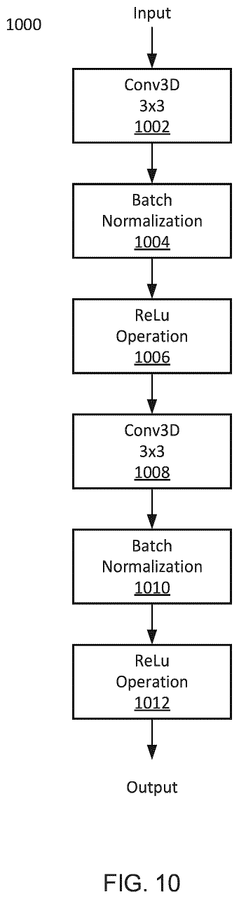

FIG. 10 shows a layout for an encoder and/or decoder 1000 disclosed herein. Each block shown in FIG. 10 may represent a logical block, such as a module, algorithm, etc. The encoder and/or decoder 1000 may comprise a first 3D convolutional logic block 1002. The first 3D convolutional logic block 1002 may comprise a 3×3 kernel. The first 3D convolutional logic block 1002 may transpose an input matrix into a first intermediate matrix. The input matrix may represent a two-dimensional (2D) image. The input matrix may represent a three-dimensional (3D) image. The input matrix may comprise dimensions of 160×128×128. The first intermediate matrix may represent a two-dimensional (2D) image. The first intermediate matrix may represent a three-dimensional (3D) image. The first intermediate matrix may comprise dimensions of 160×128×128. Other dimensions may be used.

After the first 3D convolutional logic block 1002, the first intermediate matrix may be provided to a first batch normalization logic block 1004. The first batch normalization logic block 1004 may use the first intermediate matrix to adjust normalization parameters for the first intermediate matrix and/or future input. The first batch normalization logic block 1004 may cause the first intermediate matrix to become a first normalized matrix.

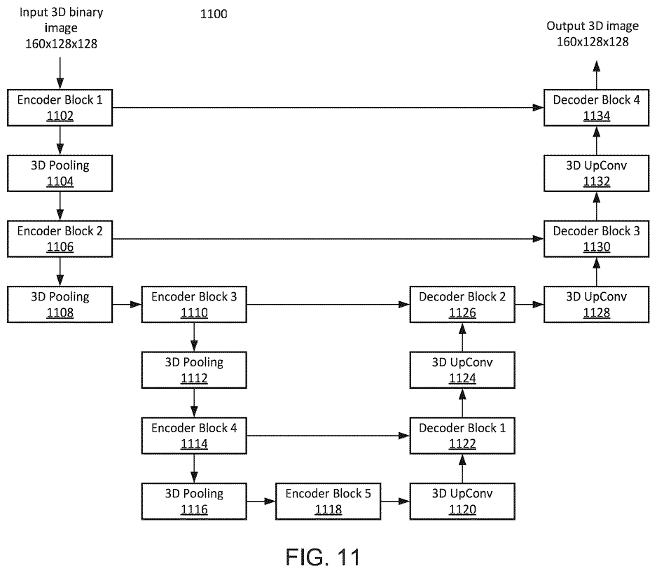

After the first batch normalization logic block 1004, the first normalized matrix may be provided to a first rectified linear unit (ReLu) operation block 1006. The first ReLu operation block 1006 may use the first normalized matrix to reenforce the encoder and/or decoder 1000 and/or an associated neural network, such as the neural network described in FIG. 11. The first ReLu operation block 1006 may cause the first normalized matrix to become a first updated matrix.

The first updated matrix may be provided as input to a second 3D convolutional logic block 1008. The second 3D convolutional logic block 1008 may comprise a 3×3 kernel. The second 3D convolutional logic block 1008 may transpose the first updated matrix into a second intermediate matrix. The first updated matrix may represent a two-dimensional (2D) image. The first updated matrix may represent a three-dimensional (3D) image. The first updated matrix may comprise dimensions of 160×128×128. The second intermediate matrix may represent a two-dimensional (2D) image. The second intermediate matrix may represent a three-dimensional (3D) image. The second intermediate matrix may comprise dimensions of 160×128×128. Other dimensions may be used.

After the second 3D convolutional logic block 1008, the second intermediate matrix may be provided to a second batch normalization logic block 1010. The second batch normalization logic block 1004 may use the second intermediate matrix to adjust normalization parameters for second first intermediate matrix and/or future input. The second batch normalization logic block 1010 may cause the second intermediate matrix to become a second normalized matrix.

After the second batch normalization logic block 1010, the second normalized matrix may be provided to a second ReLu operation block 1012. The second ReLu operation block 1012 may use the second normalized matrix to reenforce the encoder and/or decoder 1000 and/or an associated neural network, such as the neural network described in FIG. 11. The second ReLu operation block 1012 may cause the second normalized matrix to become a second updated matrix. The second updated matrix may be an output matrix. The output matrix may represent a two-dimensional (2D) image. The output matrix may represent a three-dimensional (3D) image. The output matrix may comprise dimensions of 160×128×128. Other dimensions may be used.

FIG. 11 shows an example network architecture for a convolutional neural network (CNN) 1100 disclosed herein. The CNN 1100 may comprise a plurality of encoders and decoders, like the encoder and/or decoder 1000 described in FIG. 10. The CNN 1100 may comprise 4 or 5 encoders and 4 decoders. The CNN 1100 may comprise a U shape. The CNN 1100 may comprise a plurality of 3D pooling logic blocks and a plurality of up-conversion logic blocks. The plurality of 3D pooling logic blocks and/or the plurality of up-conversion logic blocks may comprise a predetermined factor. For example, the predetermined factor may be 2. The CNN 1100 may be used to reconstruct 3D models.

The CNN 1100 may receive an image as input. The image may comprise a three-dimensional (3D) image. The image may comprise a binary image. The image may comprise a 160×128×128 matrix of voxels. A first encoder block 1102 may receive the image as input. The first encoder block 1102 may process the image to create a first processed image. The first encoder block 1102 may provide the first processed image to a first 3D pooling logic block 1104. The first encoder block 1102 may also provide the first processed image to a fourth decoder block 1134. The first 3D pooling logic block 1104 may process the first processed image to create a second processed image.

A second encoder block 1106 may receive the second processed image. The second encoder block 1106 may process the second processed image to create a third processed image. The second encoder block 1106 may provide the third processed image to a second 3D pooling logic block 1108. The second encoder block 1106 may also provide the third processed image to a third decoder block 1130. The second 3D pooling logic block 1108 may process the third processed image to create a fourth processed image.

A third encoder block 1110 may receive the fourth processed image. The third encoder block 1110 may process the fourth processed image to create a fifth processed image. The third encoder block 1110 may provide the fifth processed image to a third 3D pooling logic block 1112. The third encoder block 1110 may also provide the fifth processed image to a second decoder block 1126. The third 3D pooling logic block 1112 may process the fifth processed image to create a sixth processed image.

A fourth encoder block 1114 may receive the sixth processed image. The fourth encoder block 1114 may process the sixth processed image to create a seventh processed image. The fourth encoder block 1114 may provide the seventh processed image to a fourth 3D pooling logic block 1116. The fourth encoder block 1114 may also provide the seventh processed image to a first decoder block 1122. The fourth 3D pooling logic block 1116 may process the seventh processed image to create an eighth processed image.

A fifth encoder block 1118 may receive the eighth processed image. The fifth encoder block 1118 may process the sixth processed image to create a ninth processed image. The fifth encoder block 1118 may provide the ninth processed image to a first 3D up-conversion logic block 1120. The first 3D up-conversion logic block 1120 may process the ninth processed image to create a tenth processed image. The first 3D up-conversion logic block 1120 may provide the tenth processed image to the first decoder block 1122.

The first decoder block 1122 may process the seventh processed image and the tenth processed image to create an eleventh processed image. The first decoder block 1122 may provide the eleventh processed image to a second 3D up-conversion logic block 1124. The second 3D up-conversion logic block 1124 may process the eleventh processed image to create a twelfth processed image. The second 3D up-conversion logic block 1124 may provide the twelfth processed image to the second decoder block 1126.

The second decoder block 1126 may process the fifth processed image and the twelfth processed image to create a thirteenth processed image. The second decoder block 1126 may provide the thirteenth processed image to a third 3D up-conversion logic block 1128. The third 3D up-conversion logic block 1128 may process the thirteenth processed image to create a fourteenth processed image. The third 3D up-conversion logic block 1128 may provide the fourteenth processed image to the third decoder block 1130.

The third decoder block 1130 may process the third processed image and the fourteenth processed image to create a fifteenth processed image. The third decoder block 1130 may provide the fifteenth processed image to a fourth 3D up-conversion logic block 1132. The fourth 3D up-conversion logic block 1132 may process the fifteenth processed image to create a sixteenth processed image. The fourth 3D up-conversion logic block 1132 may provide the sixteenth processed image to the fourth decoder block 1134.

The fourth decoder block 1134 may process the first processed image and the sixteenth processed image to create a seventeenth processed image. The seventeenth processed image may be returned by the CNN 1100 as output. The seventeenth processed image may comprise a 3D image. The image may comprise a 160×128×128 matrix of voxels. Other dimension of matrices may be used.

The CNN 1100 may be implemented on a variety of platforms. For example, an example CNN 1100 may be implemented using a PyTorch library, Adam optimizer and a learning rate of 0.01. In another example, an example CNN 1100 may be implemented using a TensorFlow library. Any other library, optimizer, learning rate, etc. may be used. A loss of function of intersection over union (IOU) may be used. Training may be carried out over a predetermined number of epochs. The predetermined number may be, for example, 100.

Given a finite number of existing CT scans for training the CNN 1100, a predefined percentage of existing CT scans may be used as a training dataset and the remaining may be used for validation. For example, if 450 CT scans are used for training the CNN 1100, 90% (or 405) of the CT scans may be used to train the CNN 1100 and 10% (or 45) may be used to validate the trained CNN 1100.

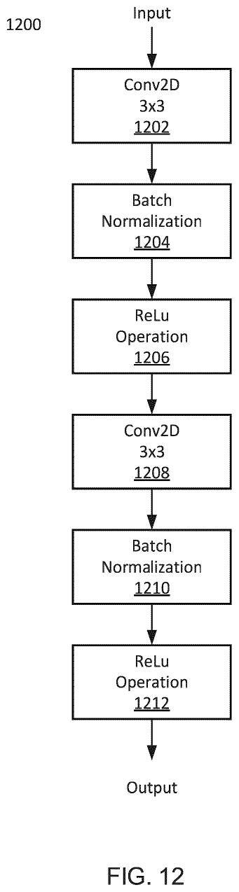

FIG. 12 shows a layout for an encoder and/or decoder 1200 disclosed herein. Each block shown in FIG. 12 may represent a logical block, such as a module, algorithm, etc. The encoder and/or decoder 1200 may comprise a first 2D convolutional logic block 1202. The first 2D convolutional logic block 1202 may comprise a 3×3 kernel. The first 2D convolutional logic block 1202 may transpose an input matrix into a first intermediate matrix. The input matrix may represent a two-dimensional (2D) image. The input matrix may represent a three-dimensional (3D) image. The input matrix may comprise dimensions of 1024×1024. The input matrix may comprise dimensions of 1024×1024×3. The input matrix may comprise a 1024×1024 sized image with 3 red-green-blue (RGB) channels. The first intermediate matrix may represent a two-dimensional (2D) image. The first intermediate matrix may represent a three-dimensional (3D) image. The first intermediate matrix may comprise dimensions of 1024×1024. The first intermediate matrix may comprise dimensions of 1024×1024×3. Other dimensions may be used.

After the first 3D convolutional logic block 1202, the first intermediate matrix may be provided to a first batch normalization logic block 1204. The first batch normalization logic block 1204 may use the first intermediate matrix to adjust normalization parameters for the first intermediate matrix and/or future input. The first batch normalization logic block 1204 may cause the first intermediate matrix to become a first normalized matrix.

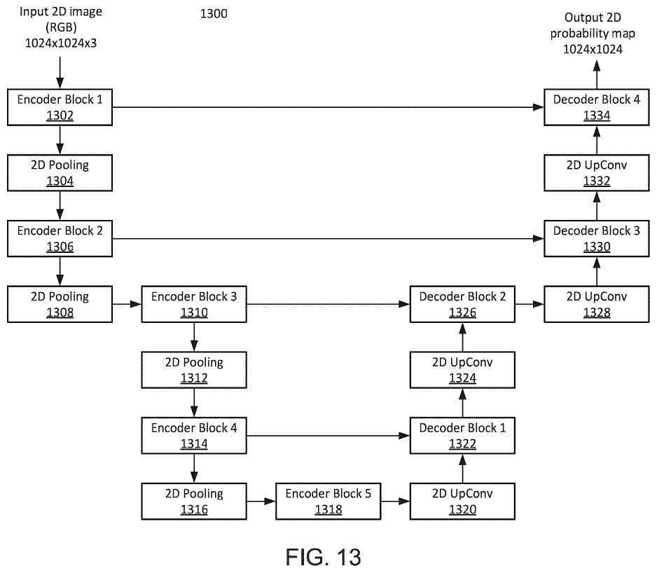

After the first batch normalization logic block 1204, the first normalized matrix may be provided to a first rectified linear unit (ReLu) operation block 1206. The first ReLu operation block 1206 may use the first normalized matrix to reenforce the encoder and/or decoder 1200 and/or an associated neural network, such as the neural network described in FIG. 13. The first ReLu operation block 1206 may cause the first normalized matrix to become a first updated matrix.

The first updated matrix may be provided as input to a second 2D convolutional logic block 1208. The second 2D convolutional logic block 1208 may comprise a 3×3 kernel. The second 2D convolutional logic block 1208 may transpose the first updated matrix into a second intermediate matrix. The first updated matrix may represent a two-dimensional (2D) image. The first updated matrix may represent a three-dimensional (3D) image. The first updated matrix may comprise dimensions of 1024×1024. The first updated matrix may comprise dimensions of 1024×1024×3. The first updated matrix may comprise a 1024×1024 sized image with 3 red-green-blue (RGB) channels. The second intermediate matrix may represent a two-dimensional (2D) image. The second intermediate matrix may represent a three-dimensional (3D) image. The second intermediate matrix may comprise dimensions of 1024×1024. The second intermediate matrix may comprise dimensions of 1024×1024×3. Other dimensions may be used.

After the second 2D convolutional logic block 1208, the second intermediate matrix may be provided to a second batch normalization logic block 1210. The second batch normalization logic block 1204 may use the second intermediate matrix to adjust normalization parameters for second first intermediate matrix and/or future input. The second batch normalization logic block 1210 may cause the second intermediate matrix to become a second normalized matrix.

After the second batch normalization logic block 1210, the second normalized matrix may be provided to a second ReLu operation block 1212. The second ReLu operation block 1212 may use the second normalized matrix to reenforce the encoder and/or decoder 1200 and/or an associated neural network, such as the neural network described in FIG. 13. The second ReLu operation block 1212 may cause the second normalized matrix to become a second updated matrix. The second updated matrix may be an output matrix. The output matrix may represent a two-dimensional (2D) probability map. Each entry (pixel, voxel, etc.) in the output matrix may comprise a value encoding a probability of the corresponding pixel in the original image belonging to an anatomical structure and/or an area of interest of an anatomical structure. The output matrix may comprise dimensions of 1024×1024. Other dimensions may be used. As referenced herein, the term pixel may be used in reference to 2D, while voxel may be referenced as 3D, and should not be read as limiting the use to only 2D or 3D.

FIG. 13 shows an example network architecture for a convolutional neural network (CNN) 1300 disclosed herein. The CNN 1300 may comprise a plurality of encoders and decoders, like the encoder and/or decoder 1200 described in FIG. 12. The CNN 1300 may comprise 4 or 5 encoders and 4 decoders. The CNN 1300 may comprise a U shape. The CNN 1300 may comprise a plurality of 2D pooling logic blocks and a plurality of up-conversion logic blocks. The plurality of 2D pooling logic blocks and/or the plurality of up-conversion logic blocks may comprise a predetermined factor. For example, the predetermined factor may be 2. The CNN may use a weighted mean square error (MSE) as accost function. The CNN 1300 may be used to create a 2D probability map, wherein each entry in the 2D probability map comprises a probability that an associated pixel of an input image corresponds to an anatomical structure and/or an area of interest of an anatomical structure.

The CNN 1300 may receive an image as input. The image may comprise a two-dimensional (2D) image. The image may comprise 3 RGB channels. The image may comprise a size of 1024 pixels by 1024 pixels. The image may comprise an ultrasound image. A first encoder block 1302 may receive the image as input. The first encoder block 1302 may process the image to create a first processed image. The first encoder block 1302 may provide the first processed image to a first 2D pooling logic block 1304. The first encoder block 1302 may also provide the first processed image to a fourth decoder block 1334. The first 2D pooling logic block 1304 may process the first processed image to create a second processed image.

A second encoder block 1306 may receive the second processed image. The second encoder block 1306 may process the second processed image to create a third processed image. The second encoder block 1306 may provide the third processed image to a second 2D pooling logic block 1308. The second encoder block 1306 may also provide the third processed image to a third decoder block 1330. The second 2D pooling logic block 1308 may process the third processed image to create a fourth processed image.

A third encoder block 1310 may receive the fourth processed image. The third encoder block 1310 may process the fourth processed image to create a fifth processed image. The third encoder block 1310 may provide the fifth processed image to a third 2D pooling logic block 1312. The third encoder block 1310 may also provide the fifth processed image to a second decoder block 1326. The third 2D pooling logic block 1312 may process the fifth processed image to create a sixth processed image.

A fourth encoder block 1314 may receive the sixth processed image. The fourth encoder block 1314 may process the sixth processed image to create a seventh processed image. The fourth encoder block 1314 may provide the seventh processed image to a fourth 2D pooling logic block 1316. The fourth encoder block 1314 may also provide the seventh processed image to a first decoder block 1322. The fourth 2D pooling logic block 1316 may process the seventh processed image to create an eighth processed image.

A fifth encoder block 1318 may receive the eighth processed image. The fifth encoder block 1318 may process the sixth processed image to create a ninth processed image. The fifth encoder block 1318 may provide the ninth processed image to a first 2D up-conversion logic block 1320. The first 2D up-conversion logic block 1320 may process the ninth processed image to create a tenth processed image. The first 2D up-conversion logic block 1320 may provide the tenth processed image to the first decoder block 1322.

The first decoder block 1322 may process the seventh processed image and the tenth processed image to create an eleventh processed image. The first decoder block 1322 may provide the eleventh processed image to a second 2D up-conversion logic block 1324. The second 2D up-conversion logic block 1324 may process the eleventh processed image to create a twelfth processed image. The second 2D up-conversion logic block 1324 may provide the twelfth processed image to the second decoder block 1326.

The second decoder block 1326 may process the fifth processed image and the twelfth processed image to create a thirteenth processed image. The second decoder block 1326 may provide the thirteenth processed image to a third 2D up-conversion logic block 1328. The third 2D up-conversion logic block 1328 may process the thirteenth processed image to create a fourteenth processed image. The third 2D up-conversion logic block 1328 may provide the fourteenth processed image to the third decoder block 1330.

The third decoder block 1330 may process the third processed image and the fourteenth processed image to create a fifteenth processed image. The third decoder block 1330 may provide the fifteenth processed image to a fourth 2D up-conversion logic block 1332. The fourth 2D up-conversion logic block 1332 may process the fifteenth processed image to create a sixteenth processed image. The fourth 2D up-conversion logic block 1332 may provide the sixteenth processed image to the fourth decoder block 1334.

The fourth decoder block 1334 may process the first processed image and the sixteenth processed image to create a seventeenth processed image. The seventeenth processed image may be returned by the CNN 1300 as output. The seventeenth processed image may comprise a 2D probability map. The size of the 2D probability map may match a size and dimension of the input image. The size of the 2D probability map may be 1024×1024. The 2D probability map may have an entry corresponding to each pixel of the input image. Each entry in the 2D probability map may comprise a probability that an associated pixel of the input image corresponds to an anatomical structure and/or an area of interest of an anatomical structure. Other dimension of matrices may be used.

The CNN 1300 may be implemented on a variety of platforms. For example, an example CNN 1300 may be implemented using a PyTorch library, Adam optimizer and a learning rate of 0.1. In another example, an example CNN 1300 may be implemented using a TensorFlow library. Any other library, optimizer, learning rate, etc. may be used. A loss of function of intersection over union (IOU) may be used. Training may be carried out over a predetermined number of epochs. The predetermined number may be, for example, 500.

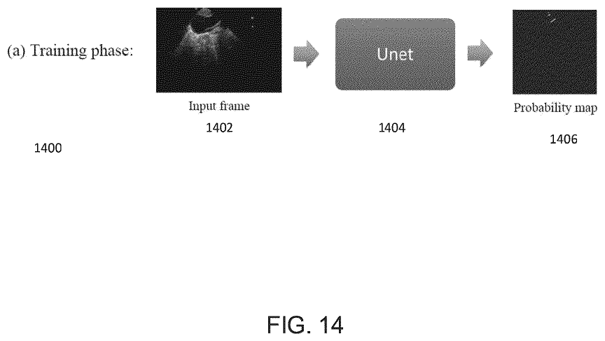

FIG. 14 shows an example training method 1400. During the example training method 1400, an image 1402 may be input into a U-shaped CNN 1404, such as the CNN 1300 in FIG. 13, and a probability map 1406 may be returned as output. The image 1402 may comprise a plurality of pixels arranged in a 2D matrix of a length and width. The image 1402 may comprise an ultrasound image. The probability map 1406 may comprise a 2D matrix comprising the length and width, and each value in the probability map 1406 may correspond to a pixel in the image 1402. A value in the probability map 1406 may indicate a probability that an associated pixel in the image 1402 corresponds to an anatomical structure and/or an area of interest of an anatomical structure.

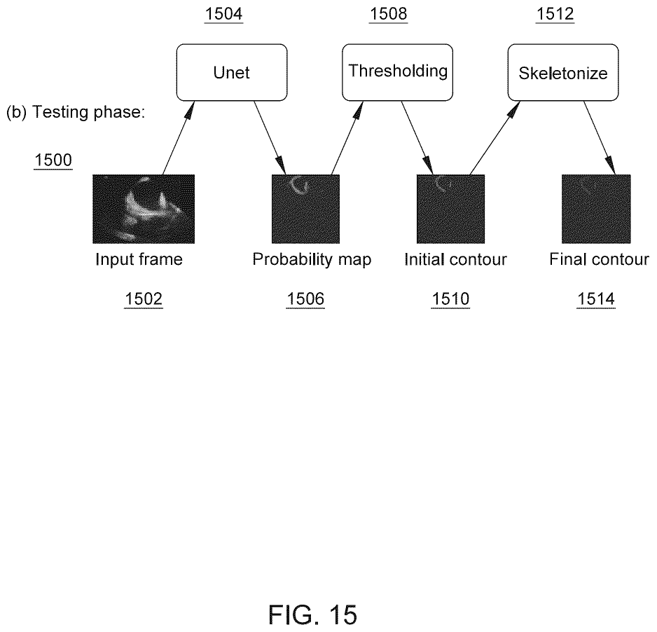

FIG. 15 shows a testing method 1500. During the example testing method 1500, an image 1502 may be input into a U-shaped CNN 1504, such as the CNN 1300 in FIG. 13, and a probability map 1506 may be returned as output. The image 1502 may comprise a plurality of pixels arranged in a 2D matrix of a length and width. The image 1502 may comprise an ultrasound image. The probability map 1506 may comprise a 2D matrix comprising the length and width, and each value in the probability map 1506 may correspond to a pixel in the image 102. A value in the probability map 1506 may indicate a probability that an associated pixel in the image 1502 corresponds to an anatomical structure and/or an area of interest of an anatomical structure. The probability map 1506 may be input into a thresholding procedure 1508. The thresholding procedure 1508 may output an initial contour 1510. The initial contour 1510 may be input into a skeletonize procedure 1512. The skeletonize procedure may output a final contour 1514. The final contour 1514 may comprise a single-pixel wide contour. As referenced herein, the term pixel may be used in reference to 2D, while voxel may be referenced as 3D, and should not be read as limiting the use to only 2D or 3D. As an example, contours may be referenced using voxel and reference to pixel herein may be read to include voxel.

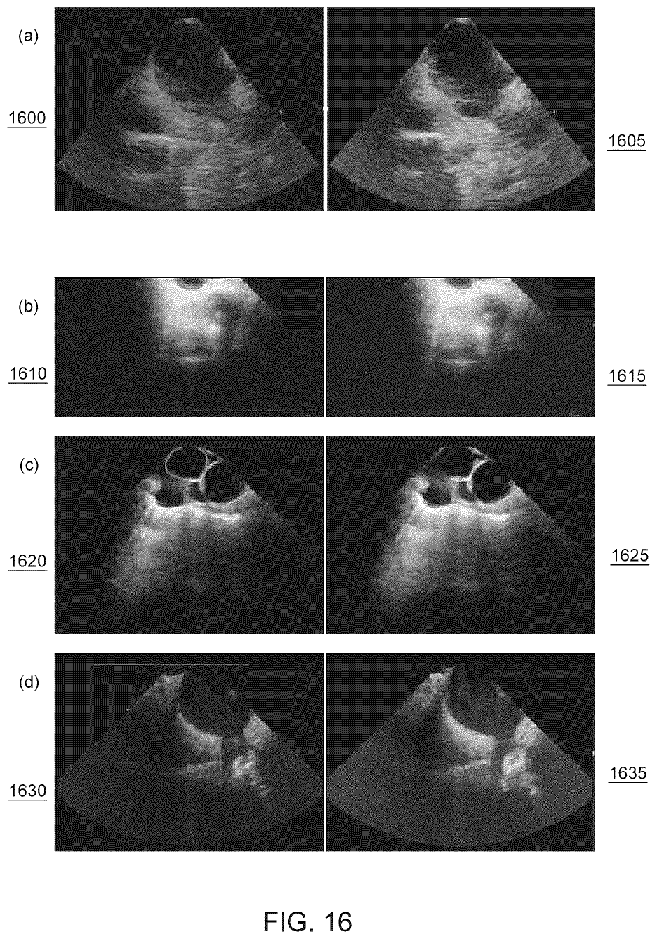

FIG. 16 shows example ultrasound images 1600, 1605, 1610, 1615, 1620, 1625, 1630, and 1635. The example images in rows may be associated, for example, 1600 and 1605 may be associated, 1610 and 1615 may be associated, 1620 and 1625 may be associated, 1630 and 1635 may be associated, etc. Images 1600, 1610, 1620, and 1630 in the left column may show an initial contour of the image highlighted. Images 1605, 1615, 1625, and 1635 in the right column may show a final contour of the image highlighted. The highlighting in image 1600 may show an initial contour of a first associated ultrasound image and the highlighting in image 1605 may show a final contour of the first associated ultrasound image, the highlighting in image 1610 may show an initial contour of a second associated ultrasound image and the highlighting in image 1615 may show a final contour of the second associated ultrasound image, the highlighting in image 1620 may show an initial contour of a third associated ultrasound image and the highlighting in image 1625 may show a final contour of the third associated ultrasound image, highlighting in image 1630 may show an initial contour of a fourth associated ultrasound image and the highlighting in image 1635 may show a final contour of the fourth associated ultrasound image, etc.

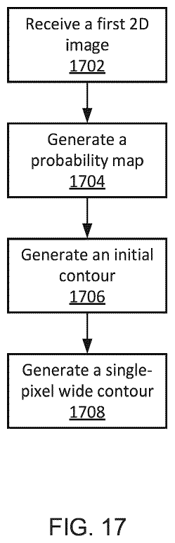

FIG. 17 is a flowchart of an example process 1700. In some implementations, one or more process blocks of FIG. 17 may be performed by a computing device. The computing device may create the final contour 1514 in FIG. 15. The computing device may be in communication with a second computing device, wherein the second computing device may create the final contour 1514 shown in FIG. 15.

A first two dimensional (2D) image may be received (block 1702). For example, a computing device may receive a first 2D image. The first 2D image may comprise a 1024×1024 matrix of pixels. The first 2D image may comprise three red-green-blue (RGB) channels. The receiving the first 2D image may comprise receiving a 3D matrix of size 1024×1024×3. The 2D image may be an ultrasound image.

A probability map may be generated (block 1704). For example, the computing device may generate a probability map. The probability map may be generated based at least on the first 2D image. Each entry in the probability map may comprise a probability associated with a likelihood of an associated pixel in the first 2D image belonging to an area of interest of an anatomical structure. The probability map may be a 2D matrix of size 1024×1024. The probability map may be generated by a model. The model may be a deep learning model. A learning rate associated with the model may be 0.1. Training of the model may be carried out for at least 500 epochs. The area of interest for the anatomical structure may be a left atrium of a heart. The anatomical structure may be a heart.

An initial contour may be generated (block 1706). For example, the computing device may generate an initial contour. The generating the initial contour may comprise thresholding the probability map. The initial contour may be generated by a convolutional neural network (CNN). The CNN may comprise a U-shape comprising five encoders and four decoders. Each encoder and decoder may comprise at least one batch normalization operation. Each encoder and decoder may comprise at least one rectified linear unit operation. Each encoder and decoder may comprise at least two batch normalization operations. Each encoder and decoder may comprise at least two rectified linear unit operations.

A single-pixel wide contour may be generated (block 1708). For example, the computing device may generate a single-pixel wide contour. The generating the single-pixel wide contour may comprise applying a skeletonize operation to the initial contour.

Although FIG. 17 shows example blocks of process 1700, in some implementations, process 1700 may include additional blocks, fewer blocks, different blocks, or differently arranged blocks than those depicted in FIG. 17. Additionally, or alternatively, two or more of the blocks of process 1700 may be performed in parallel.

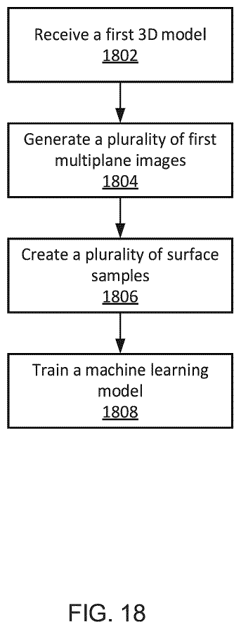

FIG. 18 is a flowchart of an example process 1800. In some implementations, one or more process blocks of FIG. 18 may be performed by a computing device. The computing device may create the images shown in FIGS. 8B-8D and 9B-9E. The computing device may be in communication with a second computing device, wherein the second computing device may create the images shown in FIGS. 8B-8D and 9B-9E.

A first three dimensional (3D) model may be received (block 1802). For example, a computing device may receive a first 3D model. The first 3D model may comprise a 160×128×128 matrix of voxels.

A plurality of first multiplane images may be generated (block 1804). For example, the computing device may generate a plurality of first multiplane images. The generating the plurality of first multiplane images may comprise generating computed tomography (CT) planes at random angles. The CT planes may be configured to simulate planes acquired by an ultrasound probe.

A plurality of surface samples may be created (block 1806). For example, the computing device may create a plurality of surface samples. The creating the plurality of surface samples may comprise sampling a portion of a CT surface for each CT plane. The portion of the CT surface may be 80% for each CT plane. The plurality of surface samples may be configured to simulate an ultrasound plane field of view.

A machine learning (ML) model may be trained (block 1808). For example, the computing device may train an ML model. The ML model may be trained to create a 3D model based on information obtained from an ultrasound probe. The ML model may be trained to create a 3D model based on data simulating contours identified in ultrasound data. The ML model may be trained to create a 3D model of a left atrium of a heart. The ML model may be trained to create a 3D model of a heart.

Although FIG. 18 shows example blocks of process 1800, in some implementations, process 1800 may include additional blocks, fewer blocks, different blocks, or differently arranged blocks than those depicted in FIG. 18. Additionally, or alternatively, two or more of the blocks of process 1800 may be performed in parallel.

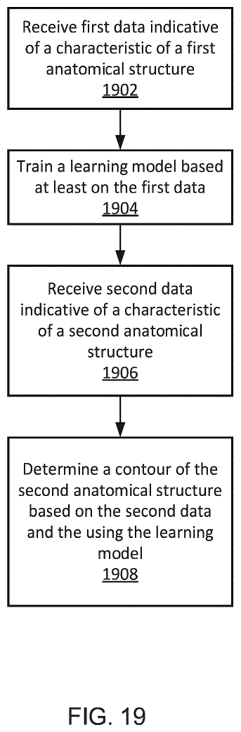

FIG. 19 illustrates an example method in accordance with the present disclosure. The method may comprises one or methods for auto contouring. The method may comprise receiving first data indicative of a characteristic of a first anatomical structure, at 1902. The first data may be or comprise CT data, which may be indicative of one or more CT scans. Other data may be used such as ULS data, or data indicative of 2D or 3D representations of structures, as described herein. A learning model, such as a deep learning AI model, may be trained based on at least the first data, at 1904. As an illustration, one or more models may be trained/tested to learn contours of certain anatomical structures based on various input data. Such data may be simulated or retrieved from actual patients. The first data may be sampled to represent a certain percentage of the anatomical structure. As described here, various processes and pre-processing may be used to train a model. Second data indicative of a characteristic of a second anatomical structure may be received, at 1906. The second data may be similar or distinct from the first data. As an example, the first data may comprise CT data, and the second data may be or comprise ULS data. Various data may be used. The second anatomical structure may be the same type of structure as the first anatomical structure, such as a heart, for example. However, the first anatomical structure may be indicative of one or more training samples of a structure, while the second anatomical structure may be associated with a specific patient. Based on the second data and the using the learning model, a contour of the second anatomical structure may be determined, at 1908. Specific implementations of one or more learning models are described herein. The contour may be outputted, for example, via a user interface.

### EXAMPLE CLAUSES

Example Clause 1: A method comprising: receiving a first two dimensional (2D) image; generating, based at least on the first 2D image, a probability map, wherein each entry in the probability map comprises a probability associated with a likelihood of an associated pixel in the first 2D image belonging to an area of interest of an anatomical structure; generating, based at least on the probability map, an initial contour; and generating, based at least on the initial contour, a single-pixel wide contour.

Example Clause 2: The method of Example Clause 1, wherein the first 2D image comprises a 1024×1024 matrix of pixels.

Example Clause 3: The method of Example Clause 1 or Example Clause 2, wherein the first 2D image comprises three red-green-blue (RGB) channels.

Example Clause 4: The method of any one of Example Clauses 1-3, wherein the receiving the first 2D image comprises receiving a 3D matrix of size 1024×1024×3.

Example Clause 5: The method of any one of Example Clauses 1-4, wherein the probability map is a 2D matrix of size 1024×1024.

Example Clause 6: The method of any one of Example Clauses 1-5, wherein the probability map is generated by a model.

Example Clause 7: The method of any one of Example Clauses 1-6, wherein the model is a deep learning model.

Example Clause 8: The method of any one of Example Clauses 1-7, wherein a learning rate associated with the model is 0.1.

Example Clause 9: The method of any one of Example Clauses 1-8, wherein training of the model is carried out for at least 500 epochs.

Example Clause 10: The method of any one of Example Clauses 1-9, wherein the generating the initial contour comprises thresholding the probability map.

Example Clause 11: The method of any one of Example Clauses 1-10, wherein the generating the single-pixel wide contour comprises applying a skeletonize operation to the initial contour.

Example Clause 12: The method of any one of Example Clauses 1-11, wherein the 2D image is an ultrasound image.

Example Clause 13: The method of any one of Example Clauses 1-12, wherein the area of interest for the anatomical structure is a left atrium of a heart.

Example Clause 14: The method of any one of Example Clauses 1-13, wherein the anatomical structure is a heart.

Example Clause 15: The method of any one of Example Clauses 1-14, wherein the initial contour is generated by a convolutional neural network (CNN).

Example Clause 16: The method of any one of Example Clauses 1-15, wherein the CNN comprises a U-shape comprising five encoders and four decoders.

Example Clause 17: The method of any one of Example Clauses 1-16, wherein each encoder and decoder comprise at least one batch normalization operation.

Example Clause 18: The method of any one of Example Clauses 1-17, wherein each encoder and decoder comprise at least one rectified linear unit operation.

Example Clause 19: The method of any one of Example Clauses 1-18, wherein each encoder and decoder comprise at least two batch normalization operations.

Example Clause 20: The method of any one of Example Clauses 1-19, wherein each encoder and decoder comprise at least two rectified linear unit operations.

Example Clause 21: A method comprising: receiving a first three dimensional (3D) model; generating, based at least on the first 3D model, a plurality of first multiplane images; creating, based at least on the plurality of first multiplane images, a plurality of surface samples; and training, based at least on the plurality of surface samples, a machine learning (ML) model.

Example Clause 22: The method of Example Clause 21, wherein the first 3D model comprises a 160×128×128 matrix of voxels.

Example Clause 23: The method of Example Clause 21 or Example Clause 22, wherein the generating the plurality of first multiplane images comprises generating computed tomography (CT) planes at random angles.

Example Clause 24: The method of any one of Example Clauses 21-23, wherein the CT planes are configured to simulate planes acquired by an ultrasound probe.

Example Clause 25: The method of any one of Example Clauses 21-24, wherein the creating the plurality of surface samples comprises sampling a portion of a CT surface for each CT plane.

Example Clause 26: The method of any one of Example Clauses 21-25, wherein the portion of the CT surface is 80% for each CT plane.

Example Clause 27: The method of any one of Example Clauses 21-26, wherein the plurality of surface samples is configured to simulate an ultrasound plane field of view.

Example Clause 28: The method of any one of Example Clauses 21-27, wherein the ML model is trained to create a 3D model based on information obtained from an ultrasound probe.

Example Clause 29: The method of any one of Example Clauses 21-28, wherein the ML model is trained to create a 3D model of a left atrium of a heart.

Example Clause 30: The method of any one of Example Clauses 21-29, wherein the ML model is trained to create a 3D model of a heart.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method for auto-contouring, the method comprising:
receiving first data indicative of a characteristic of a first anatomical structure;
training, based on at least the first data, a first learning model;
receiving second data indicative of a characteristic of a second anatomical structure; and
determining, based on the second data and using the first learning model, a contour of the second anatomical structure.

2. The method of claim 1, wherein the first data is indicative of one or more of a computer topography (CT) scan or ultrasound data.

3. The method of claim 1, wherein the first data is simulated data.

4. The method of claim 1, further comprising pre-processing the first data prior to training the first learning model, wherein the pre-processing comprises sampling a target percentage of the first data, and wherein the training the first learning model is based on the pre-processed first data.

5. The method of claim 1, wherein the second data comprises ultrasound data.

6. The method of claim 5, further comprising, generating, based at least on the contour and a second learning model, a three-dimensional (3D) model of the second anatomical structure.

7. A method for auto-contouring, the method comprising:
receiving first data indicative of a first two dimensional (2D) image;
generating, based at least on the first data, a probability map, wherein each entry in the probability map comprises a probability associated with a likelihood of an associated pixel in the first 2D image belonging to an area of interest of an anatomical structure;
generating, based at least on the probability map, an initial contour; and
generating, based at least on the initial contour, a single-pixel wide contour.

8. The method of claim 7, further comprising generating, based at least on the single-pixel wide contour and a machine learning model, a three-dimensional (3D) model of the area of interest.

9. The method of claim 7, wherein the first 2D image comprises a matrix of pixels.

10. The method of claim 7, wherein the first 2D image comprises three red-green-blue (RGB) channels.

11. The method of claim 7, wherein the receiving the first 2D image comprises receiving a 3D matrix of a first size.

12. The method of claim 11, wherein the probability map is a 2D matrix of a second size different from the first size.

13. The method of claim 7, wherein the probability map is generated by a model.

14. The method of claim 7, wherein the generating the initial contour comprises thresholding the probability map.

15. The method of claim 7, wherein the generating the single-pixel wide contour comprises applying a skeletonize operation to the initial contour.

16. The method of claim 7, wherein the 2D image is an ultrasound image.

17. The method of claim 7, wherein the area of interest for the anatomical structure is a left atrium of a heart.

18. The method of claim 7, wherein the anatomical structure is a heart.

19. The method of claim 7, wherein the initial contour is generated by a convolutional neural network (CNN).

20. The method of claim 19, wherein the CNN comprises a U-shape comprising five encoders and four decoders.

21. The method of claim 20, wherein each encoder and decoder comprise at least one batch normalization operation.

22. The method of claim 20, wherein each encoder and decoder comprise at least one rectified linear unit operation.

23. The method of claim 20, wherein each encoder and decoder comprise at least two batch normalization operations.

24. The method of claim 20, wherein each encoder and decoder comprise at least two rectified linear unit operations.

25. A method for auto-contouring, the method comprising:
receiving first data indicative of at least a portion of an anatomical structure;
generating, based at least on the first data, a probability map, wherein each entry in the probability map comprises a probability associated with a likelihood of an associated pixel or voxel belonging to an area of interest of the anatomical structure; and
generating, based at least on the probability map, a contour of the area of interest.

26. The method of claim 25, further comprising generating, based at least on the contour and a machine learning model, a three-dimensional (3D) model of the area of interest.
